(19) United States
(12) Patent Application Publication
(10) Pub. No.: US 2026/0165968 A1
(43) Pub. Date: Jun. 18, 2026

(54) SUSTAINED-RELEASE PLGA MICROSPHERE CONTAINING GS-441524, AND METHOD FOR PRODUCING SAME

(71) Applicants: THE INDUSTRY & ACADEMIC COOPERATION IN CHUNGNAM NATIONAL UNIVERSITY (IAC), Daejeon (KR); KOREA RESEARCH INSTITUTE OF CHEMICAL TECHNOLOGY, Daejeon (KR)

(72) Inventors: Cheong Weon CHO, Daejeon (KR); Hong Ki LEE, Daejeon (KR); Su Cheol HAN, Jeonju-si, Jeollabuk-do (KR); Jeong Ho HWANG, Jeonju-si, Jeollabuk-do (KR); Min Ki JIN, Daejeon (KR); Jong Hee WON, Daejeon (KR); Hye Lim YOO, Suwon-si, Gyeonggi-do (KR); Bo Min SONG, Daejeon (KR); Taek Seon YUN, Daejeon (KR); Yu Rim HWANG, Jeonju-si, Jeollabuk-do (KR)

(73) Assignees: THE INDUSTRY & ACADEMIC COOPERATION IN CHUNGNAM NATIONAL UNIVERSITY (IAC), Daejeon (KR); KOREA RESEARCH INSTITUTE OF CHEMICAL TECHNOLOGY, Daejeon (KR)

(21) Appl. No.: 19/124,507

(22) PCT Filed: Nov. 1, 2023
(86) PCT No.: PCT/KR2023/017219

§ 371 Date: Apr. 25, 2025

(30) Foreign Application Priority Data
Nov. 3, 2022 (KR) 10-2022-0145136

(51) Int. Cl. A61K 9/16; A61K 9/00; A61K 9/50

(52) U.S. Cl. CPC A61K 9/1652; A61K 9/0019; A61K 9/0024; A61K 9/0053; A61K 9/1617; A61K 9/1635; A61K 9/1647; A61K 9/5089

(57) ABSTRACT

The present invention pertains to: a sustained-release PLGA microsphere containing GS-441524; and a method for producing same. Specifically, a microsphere for sustained release within 1 week is produced by encapsulating GS-441524 in a PLGA microsphere, and thus active ingredients can be supplied stably and continuously to improve patients' medication compliance and reduce the burden of drug costs.

### TECHNICAL FIELD

The disclosure relates to sustained-release PLGA microspheres containing GS-441524 and a method for producing the same.

### BACKGROUND ART

GS-441524 is a 1′-cyano-substituted adenosine analog of remdesivir, which is a nucleoside analog antiviral agent proven to be structurally active against RNA viruses, and is named (2R,3R,4S,5R)-2-(4-aminopyrrolo[2,1-f][1,2,4]triazin-7-yl)-3,4-dihydroxy-5-(hydroxymethyl) oxolane-2-carbonitrile.

GS-441524 has been reported to be effective in treating severe acute respiratory syndrome (SARS), coronavirus, Ebola virus, and feline infectious peritonitis (FIP), and also known to inhibit the replication of various RNA viruses. However, GS-441524 is administered as a once-daily, single injection due to its half-life of about 24 hours in humans, resulting in high drug costs and frequent administrations, which may impose a burden on patients in terms of drug compliance and drug costs. Particularly, as for the treatment of feline infectious peritonitis, this analog is administered as a once-daily injection for a 12-week regimen as a basis, so that the once-daily injection for 84 days results in a painful treatment process and a great financial burden. Therefore, there is an urgent need to develop a sustained-release formulation allowing for continuous drug supply through only a single administration of GS-441524.

### DISCLOSURE OF INVENTION

### Technical Problem

An aspect of the disclosure is to provide sustained-release PLGA microspheres containing GS-441524 and a method for producing the same.

### Solution to Problem

In accordance with an aspect of the disclosure, sustained-release PLGA microspheres containing GS-441524 and a method for manufacturing the same are provided.

The disclosure provides sustained-release PLGA microspheres containing GS-441524.

Herein, GS-441524 is a 1′-cyano-substituted adenosine analog of remdesivir, which is a nucleoside analog antiviral agent proven to be structurally active against RNA viruses, and is named (2R,3R,4S,5R)-2-(4-aminopyrrolo[2,1-f][1,2,4]triazin-7-yl)-3,4-dihydroxy-5-(hydroxymethyl) oxolane-2-carbonitrile. GS-441524 is a compound having a structure of Chemical Formula 1 below.

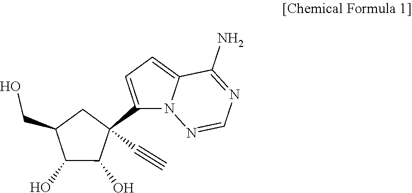 [SMILES: C#C[C@@]1(c2ccc3c(N)ncnn23)C[C@H](CO)[C@@H](O)[C@H]1O]

In the disclosure, PLGA, poly (lactic-co-glycolic acid), is a polymer where a lactic unit and a glycolic unit as monomers are crosslinked. PLGA may be a block copolymer or a random copolymer. The compositional ratio of the lactic unit and the glycolic unit according to the PLGA of the disclosure may be 50:50 to 95:5, and preferably, the compositional ratio of the lactic unit and the glycolic unit is 50:50.

In the disclosure, an organic solvent or an oil is a liquid organic substance that is capable of dissolving GS-441524 or PLGA therein and is poorly miscible with water, and examples thereof may be chloroform, ethyl ether, dichloromethane, dimethyl sulfoxide, or a mixture thereof, and specifically, may be dichloromethane or dimethyl sulfoxide, but is not limited thereto.

In the above, a stabilizer is a substance lowering the interfacial tension between a dispersed phase and an external aqueous phase, and serves to prevent the coalescence and aggregation of formed droplets through collisions. Examples thereof may include gelatin, anionic surfactants (e.g., sodium oleate, sodium stearate, sodium lauryl sulfate, and sodium dodecyl sulfate (SDS)), nonionic surfactants (e.g., polyoxyethylene sorbitan fatty esters (polysorbates), such as Tween 80 and Tween 60, and polyoxyethylene castor oil derivatives), polyvinyl alcohol (PVA), carboxymethyl cellulose, lecithin, hyaluronic acid, or a mixture thereof, and specifically, may be polyvinyl alcohol.

The microspheres according to the disclosure may be composed of 16-25 wt % of GS-441524 and 75-84 wt % of PLGA. In cases where GS-441524 is lower than 16 wt % or higher than 25 wt %, the release rate of GS-441524 from the microparticles may be too slow or too fast. The microspheres according to the disclosure may be composed of, preferably, 25 wt % of GS-441524 and 75 wt % of PLGA.

The PLGA may have a molecular weight of 7000 to 13000. In cases where the molecular weight of the PLGA is smaller than 7000 or more than 13000, the drug release rate may be faster or delayed compared with the targeted time. The ratio of the lactic unit and the glycolic unit in the PLGA is 50:50. The drug release is delayed with an increase in the proportion of the lactic unit in the ratio between the lactic unit and the glycolic unit.

The sustained-release PLGA microspheres may have a particle size of 200 to 250 nm. When the sustained-release PLGA microspheres has a particle size larger than 250 nm, the microspheres may be unstable and may be difficult to store. Preferably, the particle size of the sustained-release PLGA microspheres may be 205 to 220 nm.

Herein, the sustained-release refers to the gradual release of a drug over a long period of time within the body through the control of the drug release mechanism. Specifically, the sustained-release of the disclosure is not only controlled by simple diffusion according to the general concentration gradient (Fick's law), but may also result from a combination of the simple diffusion with a drug release mechanism by the dissolution control effect of a polymer matrix (Higuchi model). In the disclosure, the injected drug may be released by 35-60% on Day 1 and 72-95% after 7 days.

The disclosure provides a GS-441524-containing sustained-release PLGA microspheres, wherein in the microspheres, 90% or more of GS-441524 relative to the amount of injection is released for 7 days in vivo.

The GS-441524-containing sustained-release PLGA microspheres have a particle size of 200 to 250 nm, and thus can be applied as a stable injectable dosage form.

The disclosure provides a composition for treating an RNA viral infection, the composition containing GS-441524-containing sustained-release PLGA microspheres.

In the disclosure, the RNA viral infection refers to an infection caused by a virus with RNA as a gene, such as influenza virus, corona virus, reovirus, and retrovirus, and specifically, may be an infection caused by HIV virus, Ebola virus, influenza virus, corona virus, or feline infectious peritonitis-causing virus, and more specifically, may be an infection caused by any one RNA virus selected from the group consisting of SARS-COV viruses, SARS-COV-2 viruses, MERS-COV viruses, Ebola viruses, and feline infectious peritonitis-causing viruses.

The disclosure may provide a pharmaceutical composition for preventing or treating an RNA viral infection, the pharmaceutical composition containing GS-441524-containing sustained-release PLGA microspheres and a pharmaceutically acceptable excipient.

The GS-441524-containing sustained-release PLGA microspheres may be added in a content of preferably 0.001-83 wt %, more preferably 0.001-80 wt %, and most preferably 0.001-75 wt %, relative to the total weight of the entire pharmaceutical composition.

The pharmaceutical composition may be formulated in an oral dosage form, such as a powder, granules, a tablet, a capsule, a suspension, an emulsion, a syrup, a liquid, or an aerosol, or in the form of a topical preparation, a suppository, and a sterile injectable solution, according to a commonly used method for each form. Examples of a carrier, an excipient, and a diluent that may be contained in the pharmaceutical composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and a mineral oil. The pharmaceutical composition, when prepared into a formulation, may be formulated using a diluent or an excipient, such as a filler, an extender, a binder, a humectant, a disintegrant, a surfactant, a sweetener, or an acidifier, which are commonly used. Examples of solid formulations for oral administration include a tablet, a pill, a powder, granules, a capsule, and the like. These solid formulations may be formulated by mixing the GS-441524-containing sustained-release PLGA microspheres of the disclosure with at least one excipient, for example, starch, calcium carbonate, sucrose or lactose, gelatin, or the like. In addition to simple excipients, lubricants, such as magnesium stearate and talc, may be used. Examples of liquid formulations for oral administration correspond to a suspension, an oral liquid preparation, an emulsion, a syrup, and the like, and these liquid preparations may include simple diluents that are frequently used, such as water and liquid paraffin, as well as several types of excipients, such as a humectant, a sweeter, a flavoring agent, a preservative, and an acidifier. Examples of formulations for parenteral administration include a sterile aqueous solution, a non-aqueous solvent, a suspension, an emulsion, a freeze-dried preparation, and a suppository. Examples of the non-aqueous solvent and the suspension may include propylene glycol, polyethylene glycol, a vegetable oil such as olive oil, an injectable ester such as ethyl oleate, and the like. Examples of a substrate for the suppository may include Witepsol, Macrogol, Tween-61, cacao butter, laurin butter, glycerogelatin, and the like.

The dose of the pharmaceutical composition of the disclosure may vary depending on the age, sex, and body weight of a subject to be treated, the particular disease or pathological condition to be treated, the severity of a disease or pathological condition, the route of administration, and the judgment of a prescriber. The determination of the dose based on those factors is within the level of a person skilled in the art, and the general dose is in the range of approximately 0.01 mg/kg/day to 500 mg/kg/day. The dose is preferably 0.1 mg/kg/day to 200 mg/kg/day, and preferably 1 mg/kg/day to 200 mg/kg/day. The administration may be carried out once or several times in divided doses per day. The dose is not intended to limit the scope of the disclosure in any aspect.

The pharmaceutical composition of the disclosure may be administered to mammals, such as mice, livestock, and humans, through various routes. All modes of administration may be considered. For example, the administration may be performed orally, rectally, or intravenous, intramuscular, subcutaneous, intrauterine, intradural, or intracerebrovascular injection, or through application to the skin, preferably by injection.

The disclosure provides a method for manufacturing sustained-release PLGA microspheres containing GS-441524, the method including:
(1) dissolving GS-441524, which is pharmaceutically usable, in an organic solvent to prepare an organic phase;
(2) emulsifying the organic phase in step (1) in an oil phase containing PLGA to prepare an S/O emulsion;
(3) emulsifying the S/O emulsion in step (2) in an aqueous phase containing a stabilizer to prepare an S/O/W emulsion; and
(4) evaporating the organic phase and the oil phase from the S/O/W emulsion in step (3), followed by freeze-drying, to produce GS-441524-containing PLGA microspheres.

The S/O/W emulsion may be composed of 2-14 wt % of GS-441524, 8-54 wt % of PLGA, and 32-90 wt % of a stabilizer, and preferably, the S/O/W emulsion may be composed of 3-15 wt % of GS-441524, 9-35 wt % of PLGA, and 50-88 wt % of a stabilizer.

The stabilizer may be polyvinyl alcohol (PVA) having a molecular weight of 30000-70000. When the molecular weight of the polyvinyl alcohol is less than 30000 or more than 70000, the particle size of the microspheres may be smaller or larger than 250 nm and the stability of the microspheres may deteriorate, resulting in poor applicability of formulations, such as an injection.

The microspheres produced by method for manufacturing GS-441524-containing sustained-release PLGA microspheres may be composed of 25 wt % of GS-441524 and 75 wt % of PLGA, and may release a cumulative 35-60% of GS-441524 on Day 1 and a cumulative 72-95% of GS-441524 on Day 7.

### Advantageous Effects of Invention

The disclosure is directed to GS-441524-containing sustained-release PLGA microspheres and a method for manufacturing the same, and specifically, microspheres capable of sustained release of a cumulative 72-95% of GS-441524 within one week by encapsulating GS-441524 in the PLGA microspheres can be provided to achieve a stable and continuous supply of an active ingredient, thereby improving the drug compliance of patients and reducing the burden of drug costs.

### BRIEF DESCRIPTION OF DRAWINGS

FIG. 1 presents graphs showing in-vitro drug release patterns of Examples 1 to 13 (F1 to F13) according to the disclosure.

FIG. 2 presents graphs showing cumulative drug release rates on Day 1 (A) and on Day 7 (B) of Examples 1 to 13 (F1 to F13) according to the disclosure.

FIG. 3 presents a graph showing in-vitro drug release patterns of F1, F2, and F3 microspheres manufactured using PLGA 75:25.

FIG. 4 presents a scanning electron microscopic image identifying the formation of the microspheres of Example 3 (F3) according to the disclosure.

FIG. 5 presents a graph showing differential scanning calorimetry results of GS-441524, polyvinyl alcohol (PVA) as a stabilizer, a raw material of PLGA, GS-441524 containing PLGA microspheres (PLGA NPs (F3)), and GS-441524-not containing PLGA microspheres (Blank-PLGA NPs).

FIG. 6 presents a graph showing infrared spectroscopy results of GS-441524, polyvinyl alcohol (PVA) as a stabilizer, a raw material of PLGA, GS-441524 containing PLGA microspheres (PLGA NPs (F3)), and GS-441524-not containing PLGA microspheres (Blank-PLGA NPs).

FIG. 7 presents a graph showing X-ray diffraction results of GS-441524, polyvinyl alcohol (PVA) as a stabilizer, a raw material of PLGA, GS-441524 containing PLGA microspheres (PLGA NPs (F3)), and GS-441524-not containing PLGA microspheres (Blank-PLGA NPs).

FIG. 8 presents a graph showing cytotoxicity evaluation results of GS-441524, GS-441524 containing PLGA microspheres (PLGA NPs), and GS-441524-not containing PLGA microspheres (Blank-PLGA NPs).

FIG. 9 presents a standard calibration curve of GS-441524 in LC-MS/MS for quantitative analysis of GS-441524.

FIG. 10 presents graphs the concentration change of GS-441524 over time after the subcutaneous injection of Example 3 (F3, PLGA-NP) and a control (GS-441524) into rats.

### BEST MODE FOR CARRYING OUT INVENTION

The present inventors conducted research for developing a sustained release formulation of GS-441524, and as a result, the present inventors developed PLGA microspheres containing GS-441524 and confirmed the sustain release of GS-441524 within a test tube for one week, and further developed PLGA microspheres capable of sustained release in vitro for 5 days and completed the disclosure.

Hereinafter, preferable exemplary embodiments of the disclosure will be described in detail. However, the disclosure is not limited to exemplary embodiments described herein and may be embodied in other forms, and exemplary embodiments introduced herein are provided to sufficiently convey the spirit of the disclosure.

### Test Example 1. Manufacture of Microspheres According to Amount of PLGA, Proportion of Stabilizer, and Ratio of Aqueous Phase to Oil Phase

### 1.1 Manufacture of Microspheres (S/O/W Form)

Under the conditions in Table 1, 75-125 mg of PLGA was completely dissolved in 6 mL of dichloromethane (DCM) to prepare an oil phase, and 25 mg of GS-441524 was dissolved in 3 mL of dimethyl sulfoxide (DMSO) to prepare an organic phase. Then, the organic phase was added to the oil phase, followed by emulsification through an ultrasonic disperser, thereby preparing an S/O emulsion. The prepared S/O emulsion was added dropwise to 12-36 mL of an aqueous phase containing 0.5-3% of a predetermined polyvinyl alcohol (stabilizer) at a ratio of an aqueous phase to an oil phase shown in Table 1 below, followed by emulsification through an ultrasonic disperser, thereby preparing an S/O/W emulsion. Thereafter, the prepared S/O/W emulsion was stirred at 700 rpm for 12 hours in a magnetic stirring machine to cure while an organic phase and an oil phase were evaporated, and the cured product was centrifuged at 15000 rpm for 12 minutes, thereby obtaining pellets containing microspheres. Thereafter, the pellets containing microspheres thus obtained were washed with distilled water three times, frozen at −70° C., and then dried through a freeze dryer for 24 hours, thereby manufacturing PLGA microspheres containing GS-441524. Since the stabilizer was mostly removed during the washing with distilled water, the microspheres after drying contained GS-441524 and PLGA.

TABLE 1

| | | | Amount of PVA | |
|---|---|---|---|---|
| | Amount of | Amount of | | Amount of |
| | GS-441524 | PLGA | Proportion of | aqueous |
| Example | (mg/3 mL) | (mg/6 mL) | PVA (w/v %) | phase (mL) |
| 1 (F1) | 25 | 75 | 0.5 | 24 |
| 2 (F2) | 25 | 75 | 1.75 | 12 |
| 3 (F3) | 25 | 75 | 1.75 | 36 |
| 4 (F4) | 25 | 75 | 3 | 24 |
| 5 (F5) | 25 | 100 | 0.5 | 12 |
| 6 (F6) | 25 | 100 | 0.5 | 36 |
| 7 (F7) | 25 | 100 | 1.75 | 24 |
| 8 (F8) | 25 | 100 | 3 | 12 |
| 9 (F9) | 25 | 100 | 3 | 36 |
| 10 (F10) | 25 | 125 | 0.5 | 24 |
| 11 (F11) | 25 | 125 | 1.75 | 12 |
| 12 (F12) | 25 | 125 | 1.75 | 36 |
| 13 (F13) | 25 | 125 | 3 | 24 |

### 1.2 Particle Size and Drug Encapsulation Rate of PLGA Microspheres

The PLGA microspheres according to Examples 1 to 13 (F1 to F13) on Table 1 were investigated for the particle size and drug encapsulation rate of the PLGA microspheres. In the manufacturing procedure in Test Example 1.1, the drug encapsulated in the GS-441524-containing PLGA microspheres was extracted through dimethyl sulfoxide (DMSO), followed by centrifugation, and then the supernatant was taken and quantified by an LC-MS/MS system, thereby calculating the encapsulation efficiency through the following equation. The calculation results are shown in Table 2.

$$Encapsulation efficiency ( \% ) = amount of drug contained in particles / amount of drug used in the manufacture of particles \times 100 \qquad [ Equation 1 ]$$

Additionally, the microspheres manufactured under the conditions as in Examples 1-13 (F1-F13) and then freeze-dried were dispersed in distilled water, and then the average particle size of the microspheres was measured by a particle size analyzer. The measurement results are shown in Table 2.

TABLE 2

| | Particle size | Drug encapsulation | Drug release up to Day 7 |
|---|---|---|---|
| Example | (nm) | rate (%) | (%) |
| F1 | 209.8 ± 9.1 | 72.6 ± 1.3 | 91.5 ± 1.1 |
| F2 | 214.6 ± 6.6 | 67.1 ± 3.4 | 95.7 ± 1.8 |
| F3 | 215.8 ± 5.1 | 77.7 ± 0.8 | 92.4 ± 3.8 |
| F4 | 218.9 ± 1.9 | 75.1 ± 2.7 | 90.2 ± 1.7 |
| F5 | 222.9 ± 3.3 | 72.6 ± 1.0 | 83.4 ± 1.7 |
| F6 | 226.8 ± 4.8 | 80.7 ± 0.9 | 83.5 ± 1.8 |
| F7 | 223.4 ± 1.5 | 75.8 ± 1.6 | 85.0 ± 1.8 |
| F8 | 228.7 ± 3.3 | 75.4 ± 1.2 | 79.7 ± 1.6 |
| F9 | 230.3 ± 7.5 | 81.7 ± 1.3 | 76.8 ± 4.4 |
| F10 | 231.9 ± 6.9 | 80.6 ± 0.9 | 73.5 ± 0.2 |
| F11 | 230.3 ± 7.4 | 79.7 ± 1.3 | 77.2 ± 1.7 |
| F12 | 234.4 ± 7.5 | 86.0 ± 2.1 | 72.0 ± 4.0 |
| F13 | 240.8 ± 6.3 | 83.8 ± 3.3 | 74.7 ± 3.3 |

As shown in Table 2 above, the PLGA microspheres were significantly affected by the amount of PLGA in terms of the particle size and drug release. Example F1 using the smallest amount of PLGA resulted in microspheres with a smallest particle size, 209.8 nm, and Example F12 resulted in a highest drug encapsulation efficiency of 86%.

### 1.3 In Vitro Drug Release Pattern of PLGA Microspheres

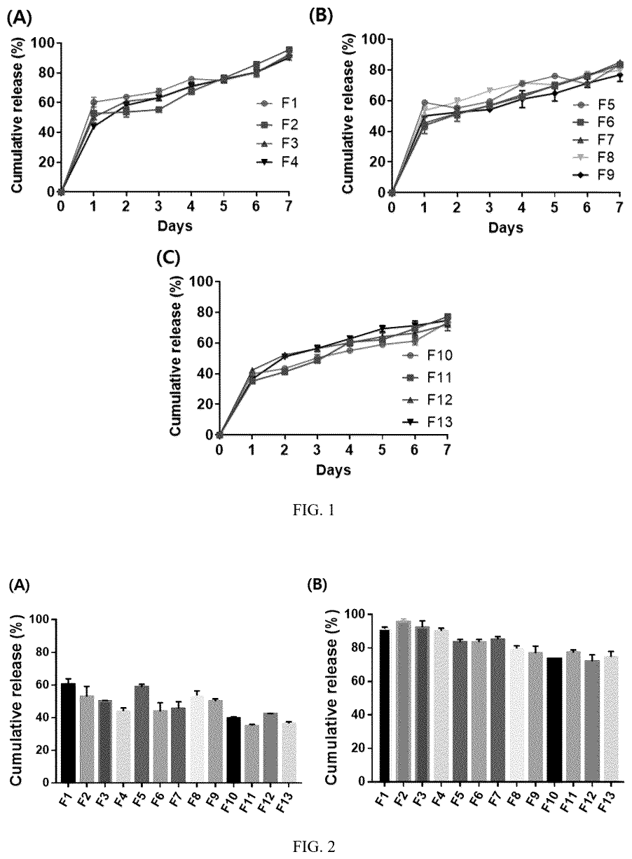

The drug release patterns of Examples 1 to 13 (F1 to F13) on Table 1 were investigated for 7 days. After 5 mg of freeze-dried PLGA microspheres of each of Examples 1 to 3 (F1 to F13) were placed in a 2 mL-tube, 1 mL of a phosphate-buffered saline (PBS) with pH 7.4 containing 1% Tween 80 as a surfactant was added and mixed, and then the mixture was used as an eluate. While the tube was stirred at 100 rpm in a shaking incubator at 37° C., 100 μL of supernatant was collected by centrifugation at a predetermined time once a day, and an equal volume of eluate was added for supplement. The concentration of GS-441524 in the collected eluate was measured by LC-MS/MS quantification, and the cumulative release amount is shown in FIG. 1. The cumulative release amounts of GS-441524 on Day 1 and on Day 7 are shown in FIGS. 2A and 2B, respectively, and for comparison, the cumulative release amount on Day 7, together with the particle size and encapsulation efficiency, is shown in Table 2.

As shown in FIGS. 1 and 2, the drug release patterns of all Examples 1 to 13 (F1 to F13) showed a drug release of 35-60% on Day 1 and 72-95% after 7 days. Particularly, Examples 1 to 4 (F1 to F4) using 75 mg of PLGA reached a cumulative release of 90% or more for 7 days. Considering the drug release results, Example 3 showing the highest encapsulation efficiency among Examples 1 to 4 showing a cumulative release of 90% or more for 7 days was used in subsequent tests.

### 1.4 In Vitro Drug Release Pattern of PLGA 75:25 Microspheres

Poly lactide-co-glycolide (PLGA), which is a synthetic polymer used in the disclosure, is a polymer compound composed of a copolymer of polylactic acid for a lactic unit and a polyglycolic acid for a glycolic unit. PLGA is used in a formulation applied in vivo due to its biodegradability and low toxicity. The PLGA used in the test examples employed a copolymer of PLA and PGA with a monomer ratio of 50:50. Therefore, it was investigated whether PLGA 75:25 also exhibited the equivalent drug release characteristics.

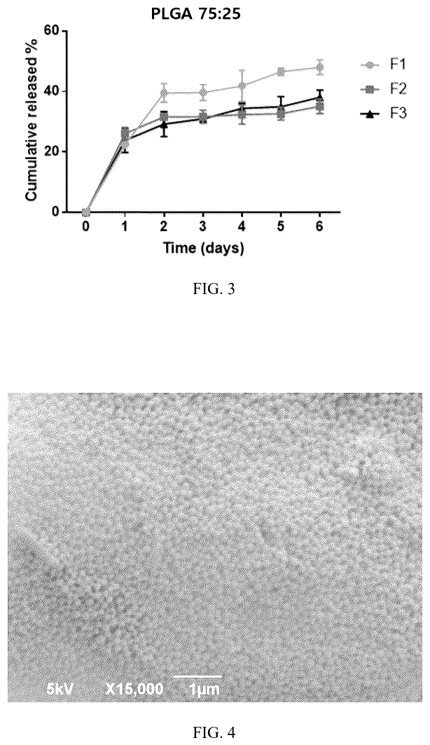

GS-441524-containing PLGA microspheres were manufactured by the same method as in Example 3 (F3) except that only PLGA was replaced with PLGA 75:25. Subsequently, the 6-day cumulative release amount of GS-441524 was measured by the same method as in Test Example 1.3, and the results are shown in FIG. 3. As shown in FIG. 3, the amount of 6-day cumulative release amount did not reach 50% under the use of PLGA 75:25, indicating that the use of PLGA 75:25 could not reach 90% even Day 7. Hence, subsequent tests were carried out using PLGA 50:50.

### Test Example 2. Evaluation of Optimized GS-441524-Containing PLGA Microspheres

The PLGA microspheres of Example 3 (F3) were manufactured using 36 mL of an aqueous phase containing 25 mg/3 mL GS-441524, 75 mg/6 mL PLGA, and 1.75 w/v % polyvinyl alcohol as a stabilizer, at an optimized composition, through Test Example 1, and formulation properties were evaluated.

### 2.1 Identification of PLGA Microsphere Formation

The PLGA microspheres of Example 3 (F3) manufactured according to the disclosure were identified by a field emission scanning electron microscope (J SM-7100F), and the results are shown in FIG. 4. As shown in FIG. 4, the manufactured microspheres of Example F3 were identified to be uniform spherical particles of about 200-250 nm.

### 2.2 Differential Scanning Calorimetry (DSC) of PLGA Microspheres

The drug encapsulation was investigated by checking the inherent melting point of a composition of the GS-441524-containing PLGA microspheres of Example 3 (F3) according to the disclosure. GS-441524, polyvinyl alcohol (PVA), and PLGA, which were used alone, and GS-441524-free PLGA microspheres (Blank-PLGA NPs), and GS-441524-containing PLGA microspheres (F3) according to the disclosure were measured for thermal transition-related temperatures by a differential scanning calorimeter (DSC N-650, SCINCO), and the results are shown in FIG. 5.

As confirmed in FIG. 5, the inherent endothermic peak of GS-441524 was observed at around 275° C., and the inherent endothermic peak of PLGA was observed at around 41° C. The PLGA microspheres of Example 3 (F3) was observed to show an endothermic peak at 48° C., and the GS-441524 contained therein was observed to show no endothermic peak. That is, after the manufacture of the PLGA microspheres, the endothermic peak of GS-441524 disappeared due to its amorphous state or the linkage with PLGA. The stabilizer polyvinyl alcohol was observed to show an endothermic peak close to that of an amorphous state at about 190° C., but such a peak was not observed in the microspheres of Example 3 (F3), indicating that the stabilizer was removed during the manufacturing steps. These results confirmed that the PLGA microspheres manufactured in the disclosure encapsulated GS-441524 in an amorphous state.

### 2.3 Fourier Transform Infrared Spectroscopy (FT-IR) of PLGA Microspheres

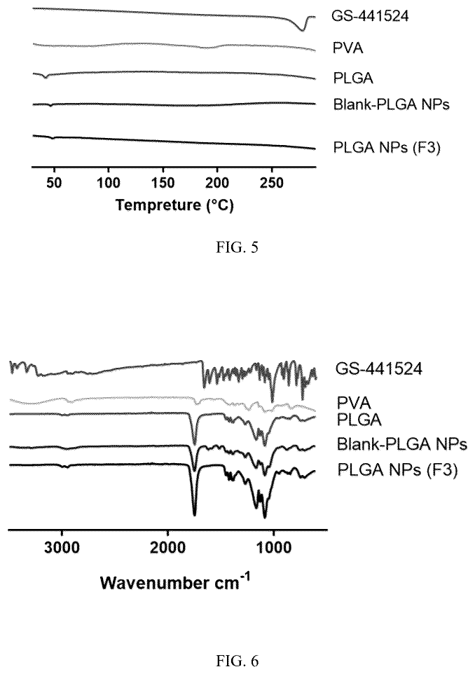

To investigate the encapsulation of the GS-441524-containing PLGA microspheres of Example 3 (F3) according to the disclosure and the interaction between the microspheres and a composition, GS-441524, polyvinyl alcohol (PVA), and PLGA, which were used alone, and GS-441524-free PLGA microspheres (Blank-PLGA NPs), and GS-441524-containing PLGA microspheres (F3) according to the disclosure were analyzed for IR spectra by an infrared spectrometer (ALPHA-P, Bruker, Massachusetts, USA), and the results are shown in FIG. 6. As shown in FIG. 6, the active ingredient, raw materials, and PLGA microsphere structure were identified, and the interaction between GS-441524 and other materials were not observed. The non-observation of the IR pattern of GS-441524 in the PLGA microspheres (F3) confirmed that GS-441524 was encapsulated in the PLGA microspheres.

### 2.4 X-Ray Diffractometry (XRD) of PLGA Microspheres

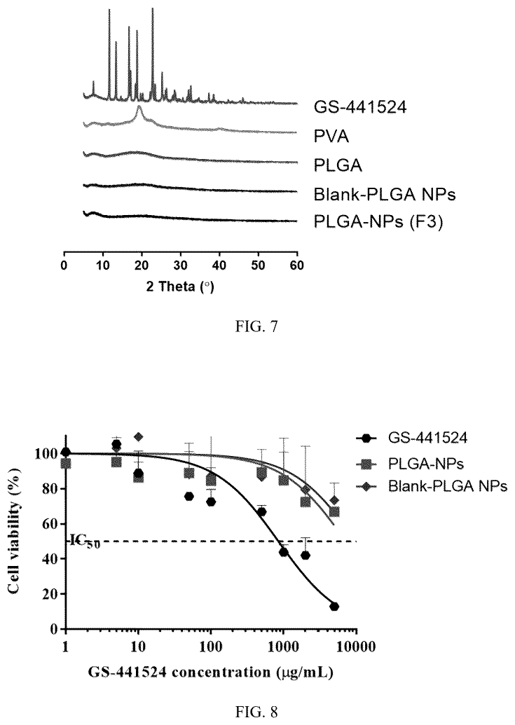

The drug encapsulation of a drug was investigated by the surface crystal morphology of the GS-441524-containing PLGA microspheres of Example 3 (F3) according to the disclosure. GS-441524, polyvinyl alcohol (PVA), and PLGA, where were used alone, and GS-441524-free PLGA microspheres (Blank-PLGA NPs), and GS-441524-containing PLGA microspheres (F3) according to the disclosure were subjected to X-ray diffractometry, and the results are shown in FIG. 7. X-ray diffractometry was conducted by a multipurpose X-ray diffractometer (D8 ADVANCE, Bruker axs, Massachusetts, USA). As confirmed in the results shown in FIG. 7, the surface of GS-441524 was measured to have a crystalline pattern. However, the PLGA microspheres (Example F3) were observed to have an amorphous pattern, indicating that GS-441524 was encapsulated in an amorphous state within the PLGA microspheres.

### Test Example 3. Evaluation of Cytotoxicity of Optimized GS-441524-Containing PLGA Microspheres

The cytotoxicity of the GS-441524-containing PLGA microspheres of Example 3 according to the disclosure was investigated.

Feline kidney cells (CRFK cells) were cultured in Dulbecco modified eagle medium (DMEM) supplemented with 10% fetal bovine serum and 1% penicillin/streptomycin (100 units/mL) under conditions of 37° C. and 5% CO2. The day before the test, CRFK cells were seeded at a density of 2×10⁴ cells per well in a 96-well plate. After one day, the medium in each well was removed, and GS-441524, GS-441524-free PLGA microspheres (Blank-PLGA NPs), or GS-441524-containing PLGA microspheres (PL GA-NPs) according to the disclosure were diluted to 5000 μM to 1 μM in media not containing an antibiotic and fetal bovine serum, and each diluted product was added to each well at 100 μL per well, and then incubated for 24 hours under conditions of 37° C. and 5% CO₂. After 24 hours, 30 μL of a 5 mg/mL thiazolyl blue tetrazolium bromide solution was added to each well, and then, the cells were cultured for 3 hours under conditions of 37° C. and 5% CO2. Thereafter, the medium in each well was removed, and then each well was washed twice with phosphate-buffered saline (PBS). Subsequently, 200 μL of dimethyl sulfoxide was added to each well, and then the absorbance was measured at an ultraviolet (UV) wavelength of 565 nm by using a plate reader. Thereafter, the results were converted to cell viability compared with the untreated control group, and the results are shown in FIG. 8.

As shown in FIG. 8, the IC50 of GS-4415234 was measured to be 849.9 μM, and the GS-441524-containing or -free PLGA microspheres showed cell viability (%) of 60% or more at even the highest drug concentration, 5000 μM, indicating that the cytotoxicity of the PL GA microspheres were significantly lower compared with GS-441524. These results may be attributed to low toxicity, which is a property of PLGA. Furthermore, the encapsulation of the active ingredient did not have a direct effect on cells, and the initial release of the PLGA microspheres (Example F3) was 50%, indicating that the drug, compared with GS-441524 alone, was applied to approximately half of the cells.

### Test Example 4. Pharmacokinetic Evaluation of GS-441524 PLGA Microspheres

The pharmacokinetic evaluation of the GS-441524-containing PLGA microspheres of Example F3 according to the disclosure was carried out. Rats as test animals were randomly divided into test and control groups, with 5 animals in each group, and raised under the same conditions while predetermined amounts of solid food and water were supplied for 7 days. The rats were fasted for 12 hours before testing, and then used for testing.

The test group was subcutaneously administered GS-441524 at a dose corresponding to 28 mg/kg by using the PLGA microspheres of Example F3 of the disclosure, and the control group was subcutaneously administered 28 mg/kg GS-441524 alone. Approximately 300 μL of blood was collected by retro-orbital bleeding 1, 2, 4, 8, 12, 24, 48, 72, 96, 120, and 144 hours after the subcutaneous injection to the rats. Each of the obtained samples was centrifuged at 15,000 rpm for 5 minutes to separate the plasma, and then stored at −70° C. before analysis.

Plasma sample pre-treatment was performed using a deproteination method. After 100 μL of a plasma sample was taken, 200 μL of methanol (the internal standard substance 10 ng/MI verapamil) was added thereto, followed by mixing for 10 minutes and then extraction. The extract was centrifuged at 17,000 rpm for 10 minutes, and then the supernatant was collected and analyzed using LC-MS/MS to quantify GS-441524 in the blood.

Table 3 below shows GS-441524 analysis conditions in LC-MS/MS, and FIG. 9 is a standard calibration curve, for quantification of GS-441524, obtained by repeating the analysis of the standard substance three times at each predetermined concentration range (0-800 ng/ml).

TABLE 3
LC-MS/MS anlaysis of GS-441524

| Equipment | Agilent Technologies 6495 Trpie Quad LC/MS | | |
|---|---|---|---|
| Column | YMC-TriartC18 column (75 × 3.0 mm, 1.9 μm) | | |
| Mobile phase | (A) 0.1% Formic acid aqueous solution | | |
| conditions | (B) 0.1% Formic acid acetonitrile | | |
| | Time(min) | A % | B % |
| | 0 | 99 | 1 |
| | 0.1 | 99 | 1 |
| | 2 | 40 | 60 |
| | 2.5 | 10 | 90 |
| | 3.5 | 10 | 90 |
| | 3.7 | 99 | 1 |
| | 4.5 | 99 | 1 |

| | |
|---|---|
| Injection volume | 5 μL |
| Flow rate | 0.4 mL/min |
| MRM | m/z 292.2 → 163.0, Positive mode |
| Column temperature | 40 |
| (° C.) | |
| Collision energy (eV) | 30 |
| Gas temperature | 290 |
| (° C.) | |
| Gas flow rate (L/min) | 18 |
| Nebulizer pressure | 40 |
| (psi) | |
| Capilary Positive | 4500 V |

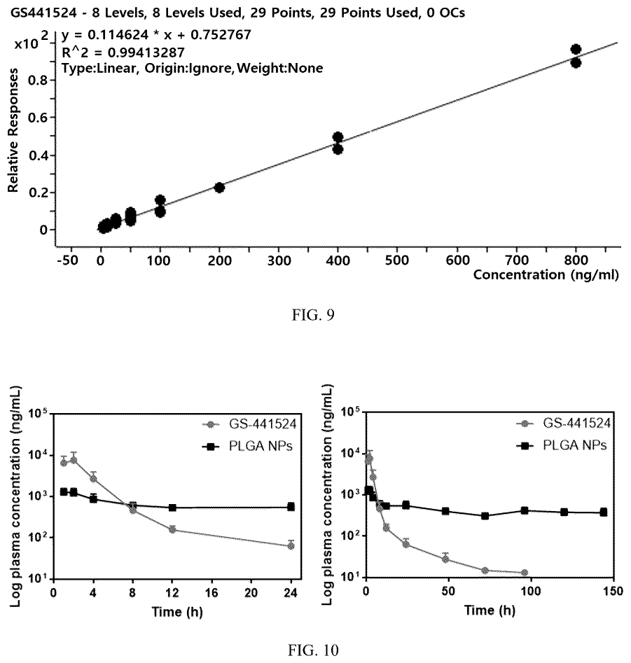

The GS-441524 concentrations in the blood thus obtained are shown in FIG. 10, and on the basis of these data, the maximum blood concentration (Cmax), area under the plasma concentration versus time curve (AUC0→144 h), half-life (T½), apparent volume of distribution (Vz), clearance (CI), and relative bioavailability (%) were calculated using WinNonlin. The results are shown in FIG. 4.

TABLE 4

| pharmacokinetic | Classification | |
|---|---|---|
| parameter | Control (GS-441524) | PLGA-NPs (Example 3) |
| Cₘₐₓ(ng/mL) | 8166 ± 4077 | 1310 ± 259 |
| AUC_{0→144}(hr · | 31615 ± 14328 | 59222 ± 13190 |
| ng/mL) | | |
| T_{1/2}(h) | 61.54 ± 66.18 | 129.62 ± 53.97 |
| Vz(mL/kg) | 81261 ± 84077 | 41419 ± 5187 |
| Cl(mL/h/kg) | 1039 ± 559 | 264 ± 146 |
| Relative bioavailability | — | 187 ± 42 |
| (%) | | |

As confirmed in Table 4 and FIG. 10, Example 3 (F3) of the disclosure, compared with the control, showed a reduction of about 53% in AUC_{0→144}, an increase in about 47% in T_{1/2}, and a reduction of about 25% in CI (clearance), confirming the sustained supply of the active ingredient GS-441524 in vivo.

1. Sustained-release PLGA microspheres containing GS-441524.

2. The sustained-release PLGA microspheres of claim 1, wherein the sustained-release PLGA microspheres release a cumulative 35-60% of GS-441524 on Day 1 and a cumulative 72-95% of GS-441524 on Day 7.

3. The sustained-release PLGA microspheres of claim 1, wherein the sustained-release PLGA microspheres are composed of 16-25 wt % of GS-441524 and 75-84 wt % of PLGA.

4. The sustained-release PLGA microspheres of claim 3, wherein the sustained-release PLGA microspheres are composed of 25 wt % of GS-441524 and 75 wt % of PLGA.

5. The composition of claim 1, wherein the PLGA has a molecular weight of 7000 to 13000, with a ratio of a lactic unit and a glycolic unit of 50:50.

6. The sustained-release PLGA microspheres of claim 1, wherein the sustained-release PLGA microspheres have a particle size of 200 to 250 nm.

7. A composition for treating an RNA viral infection, comprising the sustained-release PLGA microspheres of claim 1.

8. The composition of claim 7, wherein the RNA viral infection is caused by any one virus selected from the group consisting of SARS-COV virus, SARS-COV-2 virus, MERS-COV virus, Ebola virus, and feline infectious peritonitis (FIP)-causing virus.

9. A method for manufacturing sustained-release PLGA microspheres containing GS-441524, the method comprising:
(1) dissolving GS-441524, which is pharmaceutically usable, in an organic solvent to prepare an organic phase;
(2) emulsifying the organic phase in step (1) in an oil phase containing PLGA to prepare an S/O emulsion;
(3) emulsifying the S/O emulsion in step (2) in an aqueous phase containing a stabilizer to prepare an S/O/W emulsion; and
(4) evaporating the organic phase and the oil phase from the S/O/W emulsion in step (3), followed by freeze-drying, to produce GS-441524-containing PLGA microspheres.

10. The method of claim 9, wherein the S/O/W emulsion is composed of 2-14 wt % of GS-441524, 8-54 wt % of PLGA, and 32-90 wt % of a stabilizer.

11. The method of claim 10, wherein the S/O/W emulsion is composed of 3-15 wt % of GS-441524, 9-35 wt % of PLGA, and 50-88 wt % of a stabilizer.

12. The method of claim 9, wherein the PLGA has a molecular weight of 7000 to 13000, with a ratio of a lactic unit and a glycolic unit of 50:50.

13. The method of claim 9, wherein the stabilizer is polyvinyl alcohol (PVA) having a molecular weight of 30000-70000.

14. The method of claim 9, wherein the microspheres manufactured by the method release a cumulative 35-60% of GS-441524 on Day 1 and a cumulative 72-95% of GS-441524 on Day 7.

15. The method of claim 10, wherein the microspheres manufactured by the method release a cumulative 35-60% of GS-441524 on Day 1 and a cumulative 72-95% of GS-441524 on Day 7.

16. The method of claim 11, wherein the microspheres manufactured by the method release a cumulative 35-60% of GS-441524 on Day 1 and a cumulative 72-95% of GS-441524 on Day 7.

17. The method of claim 12, wherein the microspheres manufactured by the method release a cumulative 35-60% of GS-441524 on Day 1 and a cumulative 72-95% of GS-441524 on Day 7.

18. The method of claim 13, wherein the microspheres manufactured by the method release a cumulative 35-60% of GS-441524 on Day 1 and a cumulative 72-95% of GS-441524 on Day 7.
